SEQUENCE LISTING (6 sequences)

SEQ ID NO: 1 - protein, 5 aa, synthetic construct
  SITE 4: note = D-phenylalanine (F) in position number 4
  REGION 1..5: note = peptide RGDFK is circular
  SITE 5: note = lysine side chain links a linking group to a nucleobase polymer for lysine(K) in position number 5
RGDFK

SEQ ID NO: 2 - RNA, 41 nt, synthetic construct
  source 1..41: mol_type = other RNA
taatttctactaagtgtagatgcaatgttgttccttgagga

SEQ ID NO: 3 - DNA, 20 bp, synthetic construct
  source 1..20: mol_type = other DNA
cctcctcaaggaacaacatt

SEQ ID NO: 4 - DNA, 22 bp, synthetic construct
  source 1..22: mol_type = other DNA
gcaatgttgttccttgaggagg

SEQ ID NO: 5 - DNA, 28 bp, synthetic construct
  source 1..28: mol_type = other DNA
cctcctcaaggaacaacattgctttttt

SEQ ID NO: 6 - DNA, 85 bp, synthetic construct
  source 1..85: mol_type = other DNA
gtgtcgtgcctccgtgctgtgtttttttttttttttttttttttttttttttttttttttttttttttttttttttttttttttt

(19) United States
(12) Patent Application Publication
(10) Pub. No.: US 2026/0168007 A1
(43) Pub. Date: Jun. 18, 2026

(54) Methods and Devices for Detecting Mechanical Forces Using Cas Nuclease and Guide RNA Complexes

(71) Applicant: Emory University, Atlanta, GA (US)

(72) Inventors: Khalid Salaita, Atlanta, GA (US); Yonggang Ke, Atlanta, GA (US); Yuxin Duan, Atlanta, GA (US); Roman Sniecinski, Atlanta, GA (US)

(21) Appl. No.: 19/122,700

(22) PCT Filed: Oct. 18, 2023
(86) PCT No.: PCT/US2023/077144

§ 371 Date: Apr. 18, 2025

Related U.S. Application Data
(60) Provisional application No. 63/417,239, filed on Oct. 18, 2022.

(51) Int. Cl. C12Q 1/6818; G01N 21/64

(52) U.S. Cl. CPC C12Q 1/6818; G01N 21/6428; G01N 2021/6432; G01N 2021/6439

(57) ABSTRACT

This disclosure relates to devices and methods for signaling transient and mechanical events associated with ligand receptor interactions. In certain embodiments, this disclosure contemplates devices for detecting ligand receptor binding interactions using a pair of hybridized nucleic acids wherein a strand is conjugated to ligand and a complementary strand is conjugated to a solid support wherein upon dehybridization due to forces transmitted to the ligand-receptor complex, a surface immobilized single stranded nucleic acid is exposed and targeted by a Cas nuclease and guide RNA complex, and whereby the presence of a single stranded reporter nucleic acid is cleaved providing a signal.

### CROSS-REFERENCE TO RELATED APPLICATIONS

This application claims the benefit of U.S. Provisional Application No. 63/417,239 filed Oct. 18, 2022. The entirety of this application is hereby incorporated by reference for all purposes.

### STATEMENT REGARDING FEDERALLY SPONSORED RESEARCH OR DEVELOPMENT

This invention was made with government support under GM131099 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22098PCT.xml. The XML file is 8 KB, was created on Oct. 17, 2023, and is being submitted electronically via the USPTO patent electronic filing system.

### BACKGROUND

CRISPR/Cas systems may be engineered into vectors that function in cells. Naturally occurring Cas-crRNA complexes typically introduce a double-strand break at a specific site in bacterial DNA containing a sequence complementary to RNA, e.g., crRNA. DNA cleavage is executed by specific domains with the Cas nuclease which generate nicks on opposite DNA strands. Engineered Cas-gRNA complexes function as RNA-guided (gRNA) endonucleases with directed target sequence recognition. The gRNA and nuclease domains can be altered or mutated in order to direct specific single or double-strand breaks at desired target sequences. Protospacer-adjacent motifs (PAMs) are short sequences that improve the ability of the Cas-gRNA to complex double stranded DNA to template the target.

Chen et al. report CRISPR-Cas12a target binding unleashes indiscriminate single-stranded DNase activity. Science, 2018, 360(6387):436-439.

Xiong et al. report functional DNA regulated CRISPR-Cas12a sensors for point-of-care diagnostics of non-nucleic acid targets. JACS, 2020, 142(1):207-13.

Danhier et al., report RGD-based strategies to target alpha(v) beta(3) integrin in cancer therapy and diagnosis. Mol Pharmaceutics, 2012, 9, 2961-2973.

Ma et al. report a mechanically induced catalytic amplification reaction for readout of receptor-mediated cellular forces. Angew Chem Int Ed, 2016, 55, 5488-5492. See also US Patent Publication No. 2017/0260575.

Reference cited herein are not an admission of prior art.

### SUMMARY

This disclosure relates to devices and methods for signaling transient and mechanical events associated with ligand receptor interactions. In certain embodiments, this disclosure contemplates devices for detecting ligand receptor binding interactions using a pair of hybridized nucleic acids wherein a strand is conjugated to ligand and a complementary strand is conjugated to a solid support wherein upon dehybridization/denaturation due to forces transmitted to the ligand-receptor complex, a surface immobilized single stranded nucleic acid is exposed and targeted by a Cas nuclease and guide RNA complex, and whereby the presence of a single stranded reporter nucleic acid is cleaved providing a signal.

In certain embodiments, this disclosure relates to methods of identifying receptor ligand binding interactions comprising; contacting 1) a device comprising: a) a surface, b) a first strand of a nucleobase polymer linked to an area of the surface, c) a second strand of a nucleobase polymer configured to hybridize with the first strand and is hybridized with the first strand forming a duplex, and d) a ligand linked to the second strand; and 2) a cell comprising a receptor to the ligand applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the first strand no longer hybridizes to the second strand, the first strand becomes a single stranded nucleobase polymer; contacting the first strand with a Cas nuclease and guide RNA complex and a single stranded reporter nucleic acid, wherein the reporter nucleic acid comprises a fluorescent dye and a quencher conjugated to the reporter nucleic acid in sufficient spatial proximity such that the fluorescent dye is quenched by the quencher; wherein the guide RNA hybridizes with the first strand providing a Cas guide RNA and first strand complex, wherein the Cas guide RNA and first strand complex indiscriminately cleave the reporter nucleic acid such that the fluorescent dye is no longer in close proximity of the quencher providing a fluorescent signal; and identifying the receptor binding to a ligand by detecting the fluorescent signal as an indicator of the receptor binding to the ligand.

In certain embodiments, the Cas guide RNA and first strand complex indiscriminately cleave a single-stranded segment of the first strand releasing the Cas guide RNA and first strand complex from the surface.

In certain embodiments, the first stand comprises a fluorescent dye or quencher, wherein the second strand comprises a fluorescent dye or quencher, and wherein when the second strand is hybridized with the first strand forming a duplex the associated fluorescent dye and quencher are in sufficient spatial proximity such that the fluorescent dye is quenched by the quencher.

In certain embodiments, the first strand comprises a protospacer-adjacent motif (PAM) associated with the Cas nuclease and guide RNA complex.

In certain embodiments, contacting the device and the cell is at a temperature of greater than 20, 30, or 35 degrees Celsius.

In certain embodiments, the first strand of a nucleobase polymer is linked to an area of the surface that does not hybridize to the second strand that is of a length of less than 160, 150, or 100 nucleotides.

In certain embodiments, contacting the device and the cell is in the presence of a medium with exogenously added magnesium (Mg) salt. In certain embodiments, the medium is a growth medium. In certain embodiments, the exogenously added magnesium salt is at a concentration of greater than 1 mM, 5 mM, or 10 mM.

In certain embodiments, denaturing the duplex is by sequentially dehybridizing individual base pairs (unzipping) or shearing.

In certain embodiments, it is contemplated that the ligand comprises a polysaccharide, peptide, glycopeptide, or steroid structure. In certain embodiments, the ligand is a peptide comprising an RGD sequence. In certain embodiments, the surface is a transparent glass or polymer, and/or within a multi-well plate comprising a plurality of zones.

In certain embodiments, the Cas nuclease is Cas12a.

In certain embodiments, devices disclosed herein are used in methods of for detecting the expression or activation stage of receptors on cells, blood cells, immune cells, or platelets.

In certain embodiments, devices disclosed herein are used in methods of screening test compounds for the ability to inhibit ligand receptor interactions on cells, blood cells, immune cells, or platelets. Bold all fig and figure reference numbers

### BRIEF DESCRIPTION OF THE SEVERAL VIEWS OF THE DRAWINGS

FIG. 1A illustrates a Mechano-Cas12a Assisted Tension Sensor (MCATS) detection assay. Cellular molecular forces reveal an activator that activates Cas12a to cleave reporter strands in solution. The activator is tagged with Atto647N while the reporter DNA are tagged with Atto565 and BHQ2.

FIG. 1B shows plot data of initial cleavage rate of Cas12a over linker length of immobilized activator.

FIG. 1C shows plot data of time-fluorescent signal of Cas12a amplified signal under different temperature with 100 nM of soluble activator, 20 nM of gRNA/Cas12a complex and 100 nM reporter DNA. Results indicating Cas12a amplification provide higher signal under 37° C.

FIG. 1D shows plot data of time-fluorescent signal of Cas12a amplified signal in different buffers. Cas12a generated a relatively lower fluorescence signal in different cell culturing medium comparing with NEB buffer 2.1. Cell culture medium with additional 10 mM Mg2+ significantly boosts fluorescence signal.

FIG. 1E shows data on fluorescent signals with different amplification times when measuring human platelets tension (2*10\vec{ }6) with MCATS. Results indicate that a one-hour amplification provides better signal in cell experiments.

FIG. 2A illustrates using MCATS to detect cell forces using a plate reader to study NIH/3T3 integrin-mediated forces. Included is a schematic of DNA duplex with a concealed activator.

FIG. 2B illustrates unzipping vs shearing forces differ.

FIG. 2C shows a bar graph of MCATS signal using the same number of cells incubated on Tₜₒₗ=12 pN and Tₜₒₗ=56 pN surfaces.

FIG. 2D shows data obtained from a plate reader measuring MCATS signal as a function of Y-27632 concentration. Drug was incubated for 30 min prior to seeding. Mechano-IC₅₀ was calculated by fitting a plot to a standard dose-response function: normalized signal=100/(1+[drug]/IC₅₀). The values were normalized to the signal obtained from the 25,000 cells/well samples without drug treatment. All measurements were background subtracted using negative control wells lacking cells.

FIG. 3A illustrates a method wherein the MCATS assay is used on human platelets to determine a Mechano-IC₅₀ for antiplatelets drugs. Platelets were purified and the MCATS assay is used to study platelet mediated forces.

FIG. 3B shows plot data of individual mechano-IC₅₀, calculated with MCATS, and average and literature reported values for aspirin and eptifibatide. Each drug was incubated for 30 min prior to seeding. Mechano-IC₅₀ was calculated by fitting a plot to a standard dose-response function: Signal=Bottom+(Top-Bottom)/(1+([drug]/IC₅₀).

FIG. 3C shows plot data of plate reader measured MCATS signals as a function of 7E3 and Ticagrelor concentration.

FIG. 3D shows plot data of measured MCATS signals as a function of ADP concentration.

FIG. 4A shows a schematic workflow for using the MCATS assay to assess platelet dysfunction in CPB patients indicating MCATS is useful to predict bleeding risk for CPB patients.

FIG. 4B shows plot data of MCATS signal for healthy donor, pre-surgery, and post-surgery samples.

FIG. 4C shows plot data of platelet mechanical dysfunction (%) vs platelets transfusion needs. Dots indicate MCATS results, and each data point represents a donor.

FIG. 4D shows plot data indicating different platelet dysfunction for patients classified as moderate, severe, massive bleeding, or mild, insignificant bleeding, according to universal definition of bleeding in cardiac surgery.

FIG. 4E shows plot data of aggregometry PA (mm) for pre-CPB and post-CPB samples.

FIG. 4F shows plot data of aggregometry PA decrease (mm) vs platelets transfusion needs.

FIG. 4G shows plot data indicating aggregometry PA decreases (mm) for patients classified as moderate, severe, massive bleeding, or mild, insignificant bleeding.

FIG. 4H shows plot data of TEG MA (mm) for pre-cardiopulmonary bypass (CPB) and post-CPB samples.

FIG. 4I shows plot data of TEG MA decrease (mm) vs platelets transfusion needs.

FIG. 4J shows plot data indicating a TEG MA decrease (mm) for patients classified as moderate, severe, massive bleeding or mild, insignificant bleeding.

FIG. 5A illustrates the preparation of a fluorescent dye and quencher conjugated to oligonucleotides.

FIG. 5B illustrates the preparation of an azido substituted cyclic RGDFK (SEQ ID NO: 1) linker, wherein lysine (K) is used as the attachment point for the azido linking group.

FIG. 5C illustrates the preparation of the triazole linking group by reacting an alkynyl substituted oligonucleotide with the azido substituted cyclic RGDFK (SEQ ID NO: 1) linker, wherein the lysine (K) amino acid is used as the attachment point for the azido linking group.

FIG. 6A shows data using MACTS for CPB-related platelet dysfunction. Altered platelet function are widely known consequences of cardiopulmonary bypass (CPB). Four patients underwent cardiac surgery. Pre-CPB and post-CPB, TEG and MACTS were measured before any transfusions. MCATS suggested significant platelet dysfunction in all 4 patients, and TEG suggested the need for platelets in only 2 of the patients.

FIG. 6B shows data on the ratio of fluorogenic signal with heparin or without heparin in patients having a positive or negative serotonin-release assay (SRA). Serum was obtained from patients and sent for ELISA Heparin Induced Thrombocytopenia (HIT) testing. Serum was incubated with normal platelets with and without heparin. Patient serum that was SRA positive had increase fluorogenic signal when heparin was added.

FIG. 6C illustrates using MCATS to detect HIT. Stored plasma from hospitalized patients (n=14) who underwent ELISA and SRA testing was utilized because of suspected HIT. Donor platelets were incubated with plasma of each patient with and without heparin. The MCATS fluorogenic signal ratios with and without heparin for patients who tested positive and negative by the SRA are shown in FIG. 6B. If patient plasma had functional HIT antibodies, they combined with the heparin and activated the platelet, causing in increase in platelet force, which in turn increased the fluorogenic signal compared to baseline.

### DETAILED DESCRIPTION

Before the present disclosure is described in greater detail, it is to be understood that this disclosure is not limited to embodiments described, and as such may, of course, vary. It is also to be understood that the terminology used herein is for describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps. The term “comprising” in reference to an oligonucleotide having a nucleic acid sequence refers to an oligonucleotide that may contain additional 5′ (5′ terminal end) or 3′ (3′ terminal end) nucleotides, i.e., the term is intended to include the oligonucleotide sequence within a larger nucleic acid. “Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein. The term “consisting of” in reference to an oligonucleotide having a nucleotide sequence refers an oligonucleotide having the exact number of nucleotides in the sequence and not more or having not more than a range of nucleotide expressly specified in the claim. For example, “5′ sequence consisting of” is limited only to the 5′ end, i.e., the 3′ end may contain additional nucleotides. Similarly, a “3′ sequence consisting of” is limited only to the 3′ end, and the 5′ end may contain additional nucleotides.

As used herein, the term “cell” refers to a biological compartment containing a lipid membrane and cytosol which may contain a nucleus containing genetic material, mitochondria, and other organelles. Thrombocytes (platelets) are unique cells derived from fragments of a progenitor megakaryocyte found in bone marrow. Platelets contain a lipid membrane, mitochondria, mRNA, proteins, granules but lack a nucleus. Platelets have a characteristic discoid shape typically about 1 to 3 μm in diameter.

The terms, “cell culture” or “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide and/or optionally nucleotides. Typical components in a growth medium include amino acids (histidine, isoleucine, leucine, lysine, methionine, phenylalanine, threonine, tryptophan, valine, and others); vitamins such as retinol, carotene, thiamine, riboflavin, niacin, biotin, folate, and ascorbic acid; carbohydrate such as glucose, galactose, fructose, or maltose; inorganic salts such as sodium, calcium, iron, potassium, magnesium, zinc; serum; and buffering agents. Additionally, a growth media may contain phenol red as a pH indication. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate. Minimal Essential Medium (MEM) is a term of art referring to a growth medium that contains calcium chloride, potassium chloride, magnesium sulfate, sodium chloride, sodium phosphate and sodium bicarbonate), essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin B9), choline, and myo-inositol (originally known as vitamin B8). Various growth mediums are known in the art. Dulbecco's modified Eagle's medium (DMEM) is a growth medium which contains additional components such as glycine, serine, and ferric nitrate with increased amounts of vitamins and amino acids.

The term “conjugated” refers to linking molecular entities through covalent bonds, or by other specific binding interactions, such as due to hydrogen bonding or other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to restrict the breaking of bonds in order to implement the intended results. In certain embodiments, the term conjugated is intended to include linking molecular entities that do not break unless exposed to a force of about greater than about 5, 10, 25, 50, 75, 100, 125, or 150 pN depending on the context.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C=O)—, —(C═NH)—, —(C═S)—, —(C═CH₂)—, which may contain single, double, or triple bonds individually and independently between the R groups. If an R is branched with an Ra it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, polyethylene glycols, amides, esters, and aromatic groups.

The terms, “nucleic acid,” or “oligonucleotide,” is meant to include nucleic acids, ribonucleic or deoxyribonucleic acid, mixtures, nucleobase polymers, or analog thereof. An oligonucleotide can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine, or guanine.

The term “nucleobase polymer” refers to nucleic acids and chemically modified forms with nucleobase monomers. In certain embodiments, methods and compositions disclosed herein may be implemented with nucleobase polymers comprising units of a ribose, 2′deoxyribose, locked nucleic acids (1-(hydroxymethyl)-2,5-dioxabicyclo[2.2.1]heptan-7-ol), 2′-O-methyl groups, a 3′-3′-inverted thymidine, phosphorothioate linkages, or combinations thereof. In certain embodiments, the nucleobase polymer may be less than 100, 50, or 35 nucleotides or nucleobases.

Nucleobase monomers are nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may be single or double stranded or both, e.g., they may contain overhangs. Nucleobase polymers may contain naturally occurring or synthetically modified bases and backbones. In certain embodiments, a nucleobase polymer need not be entirely complementary, e.g., may contain one or more insertions, deletions, or be in a hairpin structure provided that there is sufficient selective binding.

With regard to the nucleobases, it is contemplated that the term encompasses isobases, otherwise known as modified bases, e.g., are isoelectronic or have other substitutes configured to mimic naturally occurring hydrogen bonding base-pairs. Examples of nucleotides with modified adenosine or guanosine include, but are not limited to, hypoxanthine, xanthine, 7-methylguanine. Examples of nucleotides with modified cytidine, thymidine, or uridine include 5,6-dihydrouracil, 5-methylcytosine, 5-hydroxymethylcytosine. Contemplated isobases include 2′-deoxy-5-methylisocytidine (iC) and 2′-deoxy-isoguanosine (iG) (see U.S. Pat. Nos. 6,001,983; 6,037,120; 6,617,106; and 6,977,161).

Nucleobase polymers may be chemically modified, e.g., within the sugar backbone or on the 5′ or 3′ ends. As such, in certain embodiments, nucleobase polymers disclosed herein may contain monomers of phosphodiester, phosphorothioate, methylphosphonate, phosphorodiamidate, piperazine phosphorodiamidate, ribose, 2′-O-methy ribose, 2′-O-methoxyethyl ribose, 2′-fluororibose, deoxyribose, 1-(hydroxymethyl)-2,5-dioxabicyclo[2.2.1]heptan-7-ol, P-(2-(hydroxymethyl)morpholino)-N,N-dimethylphosphon amidate, morpholin-2-ylmethanol, (2-(hydroxymethyl)morpholino) (piperazin-1-yl)phosphinate, or peptide nucleic acids or combinations thereof.

In certain embodiments, the nucleotide base polymer is single or double stranded and/or is 3′ end capped with one, two, or more thymidine nucleotides and/or a 5′ end that is polyphosphorylated, e.g., di-phosphate or tri-phosphate. In certain embodiments, the nucleobase polymer can be modified to contain a phosphodiester bond, methylphosphonate bond, or phosphorothioate bond. The nucleobase polymers can be modified, for example having a 2′-amino, 2′-C-allyl, 2′-fluoro, 2′-O-methyl, 2′-H on the ribose ring. Constructs can be purified by gel electrophoresis using general methods or can be purified by high pressure liquid chromatography and re-suspended in water.

In certain embodiments, nucleobase polymers include one or more (e.g., about 1, 2, 3, 4, 5, 6, 7, 8, 9, 10, or more) LNA “locked nucleic acid” nucleotides such as a 2′,4′-C methylene bicyclo nucleotide (see for example U.S. Pat. Nos. 6,639,059, 6,670,461, 7,053,207). In certain embodiments, the disclosure features modified nucleobase polymers, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “specific binding agent” refers to a molecule, such as a proteinaceous molecule, that binds a target molecule with a greater affinity than other random molecules or proteins. Examples of specific binding agents include an antibody that binds an epitope of an antigen or a receptor which binds a ligand. In certain embodiments, “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

As used herein, the term “ligand” refers to an organic molecule, i.e., substantially comprised of carbon, hydrogen, and oxygen, that binds a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin may bind the glycan. As the glycan is typically smaller and surrounded by the lectin during binding, it may be considered a ligand even though it is a receptor of the lectin binding signal on the cell surface. In another example, a double stranded oligonucleotide sequence contains two complimentary nucleic acid sequences. Either of the single stranded sequences may be considered the ligand or receptor of the other. In certain embodiments, a ligand is contemplated to be a compound that has a molecular weight of less than 500 or 1,000. In certain embodiments, a receptor is contemplated to be a compound that has a molecular weight of greater than 2,000 or 5,000. In any of the embodiments disclosed herein the position of a ligand and a receptor may be switched.

As used herein, the term “surface” refers to the outside part of an object. The area is typically of greater than about one hundred square nanometers, one square micrometer, or more than one square millimeter. Examples of contemplated surfaces are on a particle, bead, wafer, array, well, microscope slide, transparent or opaque glass, polymer, or metal, or the bottom of a zero-mode waveguide. A “zero-mode waveguide (ZMW)” refers to a confined structure or chamber located in an opening, e.g., hole, of a metal film deposited on a transparent substrate. See Levene et al., Science, 2003, 299:682-686. The chamber acts as a wave guide for light coming out of the bottom of the opening. The openings are typically about 150-50 nm in width and depth. Due to the behavior of light when it travels through a small aperture, the optical field decays exponentially inside the chamber. Thus, fluorescent molecules will lose fluorescence as they move away from the bottom of the chamber.

As used herein, “subject” refers to any animal, preferably a human patient, livestock, or domestic pet.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating,” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

### CRISPR and Cas Nucleases

The clustered regularly interspaced short palindromic repeat (CRISPR) system is a prokaryotic adaptive immune system that has been modified to enable general genome engineering in a variety of organisms and cell lines. CRISPR-Cas (CRISPR associated) systems are protein-RNA complexes that use an RNA molecule (crRNA) as a guide (gRNA segment) to localize the complex to a target nucleic acid sequence via base-pairing. In the natural systems, CRISPR associated (Cas) proteins then acts as a nuclease to cleave the targeted DNA sequence. The target sequence contains a “protospacer-adjacent motif” (PAM) oligonucleotide adjacent to the target region in order for the system to function.

Among the known Cas nucleases, Cas12a (also known as Cpf1) and S. pyogenes Cas9 has been widely reported. See e.g., GenBank accession number AJ156734.1 is reported as a 939 CRISPR-associated protein from Francisella philomiragia (Cas12a). Cas12a is a single RNA-guided endonuclease. Cas12a-associated CRISPR arrays are processed into mature crRNAs without the requirement of an additional trans-activating crRNA (tracrRNA). Cas12a-crRNA complexes efficiently cleave target DNA proceeded by a short T-rich protospacer-adjacent motif (PAM) and the target sequence is typically in the 3′ direction (downstream) from the PAM sequence. See Zetsche et al. Cpf1 is a single RNA-guided endonuclease of a class 2 CRISPR-Cas system. Cell, 2015, 163(3):759-71.

As used herein, a “guide RNA” refers to a single RNA or multiple RNAs that complex with each other containing a guide sequence for a target and other sequences that facilitate binding to a Cas nuclease.

As used herein, the term “Cas nucleases” means a protein having an ability to bind to a DNA molecule in the presence of gRNA, including Cpf1 and Cas9 proteins, having dual nuclease activities or lacking either or both the two nuclease activities. Wild-type Cas proteins have two functional endonuclease domains. One domain cleaves one strand of a double strand DNA, and the other domain cleaves another strand. When both domains are active, the Cas proteins can generate the double strand breaks in genomic DNA. Point mutations can be introduced into Cas nucleases to abolish nuclease activity, resulting in a nuclease inactive Cas nuclease that still retains its ability to bind DNA in a gRNA-programmed manner.

In certain embodiment, a Cas nuclease is a fusion protein with at least one other protein or peptide. Such proteins and peptides include, for example, fluorescent proteins, transcriptional factors, epitope tags, tags for protein purification, nuclear localization signal peptides, and transcriptional regulators. Cas nucleases mRNA may be obtained by cloning a DNA coding an amino acid sequence of a desired Cas nuclease into a vector suitable for in vitro transcription and performing in vitro transcription.

Guide RNAs come in different forms. One form uses a separate targeting guide RNA and a tracrRNA that hybridize together to guide targeting, and another, uses a chimeric targeting guide RNA-tracrRNA hybrid that links the two separate RNAs in a single strand of RNA that forms a hairpin, referred to as sgRNA. See also Jinek et al., Science 2012; 337:816-821. The tracrRNA can be variably truncated and a range of lengths has been shown to function in both forms. In the natural state, crRNA is responsible for sequence specificity of gRNA. The target sequence may be present in either strand of the genomic DNA. In certain embodiments, the gRNA comprises a sequence that is identical to the sense or template strand that is upstream or downstream from the PAM or reverse complement of the PAM. Tools are available for selecting a target sequence and/or designing gRNA and lists of target sequences which are predicted for various genes in various species may be obtained. See, e.g., Feng Zhang lab's Target Finder, Michael Boutros lab's Target Finder (E-CRISP), RGEN Tools: Cas-OFFinder, CasFinder: Flexible algorithm for identifying specific Cas targets in genomes, and CRISPR Optimal Target Finder.

Guide RNA (gRNA) may be obtained by cloning a DNA having a desired gRNA sequence into a vector suitable for in vitro transcription and performing in vitro transcription. Guide RNA may be obtained by inserting a synthesized oligonucleotide of a target sequence into such vector and performing in vitro transcription. Such commercial vectors include, for example, pUC57-sgRNA expression vector, pCFD1-dU6:1gRNA, pCFD2-dU6:2gRNA pCFD3-dU6:3gRNA, pCFD4-U6:1_U6:3tandemgRNAs, pRB17, pMB60, DR274, SP6-sgRNA-scaffold, pT7-gRNA, DR274, and pUC57-Simple-gRNA backbone available from AddgeneT^{M}, and pT7-Guide-IVT available from Origene™.

In certain embodiments, gRNA disclosed herein are capable of specifically hybridizing to the target nucleic acid. As used herein, two nucleic acid molecules are said to be capable of specifically hybridizing to one another if the two molecules are capable of forming a hydrogen bonding nucleic acid structure. A nucleic acid molecule may exhibit complete or incomplete complementarity. Departures from complete complementarity are therefore permissible, as long as such departures do not completely preclude the capacity of the gRNA molecules to form a hydrogen bonding structure with the target. Thus, in order for an RNA to serve as a guide to the target, the RNA needs only be sufficiently complementary in sequence to be able to form a stable hydrogen bonding structure under the experimental conditions, e.g., representative of physiological conditions.

### Tension Sensor Devices and Methods of Use

This disclosure relates to devices and methods for signaling transient and rare mechanical events in cells. In certain embodiments, this disclosure contemplates devices for detecting ligand receptor binding interactions using a pair of hybridized nucleic acids wherein a strand is conjugated to ligand and complementary strand is conjugated to a solid support wherein upon dehybridization due to forces associated with ligand receptor binding interactions, the surface immobilized single stranded nucleic acid is exposed and targeted by a Cas nuclease and guide RNA complex, and whereby the presence of a single stranded reporter nucleic acid is cleaved providing a signal.

In certain embodiments, this disclosure relates to methods of identifying receptor ligand binding interactions comprising; contacting 1) a device comprising: a) a surface, b) a first strand of a nucleobase polymer linked to an area of the surface, c) a second strand of a nucleobase polymer configured to hybridize with the first strand and is hybridized with the first strand forming a duplex, and d) a ligand linked to the second strand; and 2) a cell comprising a receptor to the ligand applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the first strand no longer hybridizes to the second strand, the first strand becomes a single stranded nucleobase polymer; contacting the first strand with a Cas nuclease and guide RNA complex and a single stranded reporter nucleic acid, wherein the reporter nucleic acid comprises a fluorescent dye and a quencher conjugated to the reporter nucleic acid in sufficient spatial proximity such that the fluorescent dye is quenched by the quencher; wherein the guide RNA hybridizes with the first strand providing a Cas guide RNA and first strand complex, wherein the Cas guide RNA and first strand complex indiscriminately cleave the reporter nucleic acid such that the fluorescent dye is no longer in close proximity of the quencher providing a fluorescent signal; and identifying the receptor binding to a ligand by detecting the fluorescent signal as an indicator of the receptor binding to the ligand.

In certain embodiments, the Cas guide RNA and first strand complex indiscriminately cleave a single-stranded segment of the first strand releasing the Cas guide RNA and first strand complex from the surface.

In certain embodiments, the first stand comprises a fluorescent dye or quencher, wherein the second strand comprises a fluorescent dye or quencher, and wherein when the second strand is hybridized with the first strand forming a duplex the associated fluorescent dye and quencher are in sufficient spatial proximity such that the fluorescent dye is quenched by the quencher.

In certain embodiments, the first strand comprises a protospacer-adjacent motif (PAM) associated with the Cas nuclease and guide RNA complex.

In certain embodiments, it is contemplated that the ligand comprises a polysaccharide, peptide, glycopeptide, or steroid structure. In certain embodiments, the peptide comprises an RGD sequence. In certain embodiments, the peptide is of the following formula:

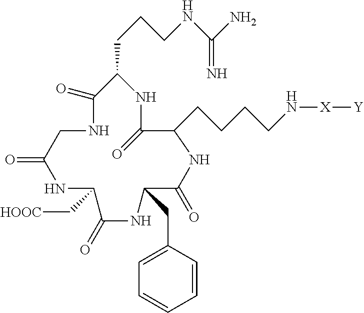

wherein X is a linking group Y is the second strand nucleobase polymer.

In certain embodiments, this disclosure relates to compositions of matter comprising the RGD peptide having the following formula:

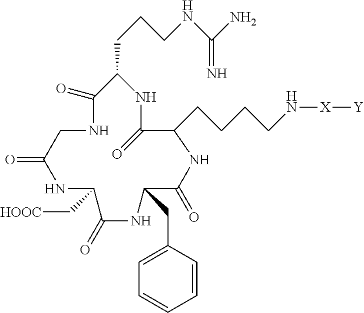

or salt thereof, wherein X is a linking group and Y is a nucleobase polymer.

In certain embodiments, the surface is a transparent glass or polymer. In certain embodiments, the device has a surface comprising gold nanoparticles wherein the first stand of nucleobase polymer is linked to the gold nanoparticles.

In certain embodiments, the Cas nuclease is Cas12a.

In certain embodiments, the receptor comprises a polypeptide of ten or more amino acids.

In certain embodiments, devices disclosed herein are used in methods of for detecting the expression or activation stage of receptors on cells, blood cells, immune cells, or platelets.

In certain embodiments, devices disclosed herein are used in methods of screening test compounds for the ability to inhibit ligand receptor interactions on cells, blood cells, immune cells, or platelets.

In certain embodiments, this disclosure relates to methods of identifying a receptor binding to a ligand comprising; contacting 1) a device comprising: a) a surface, b) a first strand of a nucleobase polymer linked to an area of the surface, c) a second strand of a nucleobase polymer configured to hybridize with the first strand and is hybridized with the first strand forming a duplex, and d) a ligand linked to the second strand; and 2) a cell comprising a receptor to the ligand applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the first strand no longer hybridizes to the second strand, the first strand becomes a single stranded nucleobase polymer; contacting the first strand with a Cas nuclease and guide RNA complex and a single stranded reporter nucleic acid, wherein the reporter nucleic acid comprises a fluorescent dye and a quencher conjugated to the reporter nucleic acid in sufficient spatial proximity such that the fluorescent dye is quenched by the quencher; wherein the guide RNA hybridizes with the first strand providing a Cas guide RNA and first strand complex, wherein the Cas guide RNA and first strand complex indiscriminately cleave the reporter nucleic acid such that the fluorescent dye is no longer in close proximity of the quencher providing a fluorescent signal; and identifying the receptor binding to a ligand by detecting the fluorescent signal as an indicator of the receptor binding to the ligand.

In certain embodiments, the Cas guide RNA and first strand complex cleave a single-stranded segment of the first strand de-conjugating the complex from the surface.

In certain embodiments, the first stand comprises a fluorescent dye or quencher, wherein the second strand comprises a fluorescent dye or quencher, and wherein when the second strand is hybridized with the first strand forming a duplex the associated fluorescent dye and quencher are in sufficient spatial proximity such that the fluorescent dye is quenched by the quencher.

In certain embodiments, the first strand comprises a protospacer-adjacent motif (PAM) associated with the Cas nuclease and guide RNA complex.

In certain embodiments, the ligand comprises a polysaccharide, peptide, glycopeptide, or steroid structure.

In certain embodiments, the Cas nuclease is Cas12a.

In certain embodiments, the surface is a transparent glass or polymer.

In certain embodiments, contacting the device and the cell is at a temperature of greater than 20, 30, or 35 degrees Celsius.

In certain embodiments, the first strand of a nucleobase polymer is linked to an area of the surface that does not hybridize to the second strand that is of a length of less than 160, 150, or 100 nucleotides.

In certain embodiments, contacting the device and the cell is in the presence of a medium with exogenously added magnesium (Mg) salt. In certain embodiments, the medium is a growth medium. In certain embodiments, the exogenously added magnesium salt is at a concentration of greater than 1 mM, 5 mM, or 10 mM.

In certain embodiments, denaturing the duplex is by sequentially dehybridizing individual base pairs (unzipping) or non-sequential dehybridization (shearing).

In certain embodiments, methods disclosed herein further comprise recording the fluorescent signal on a computer readable medium (non-transient storage medium) providing a background signal, adding a test compound to the device, detecting the fluorescent signal on a computer readable medium providing a test compound signal, and comparing the background signal to the test compound signal.

In certain embodiments, method of identifying the effect of a test compound to inhibit a receptor binding to a ligand comprising; contacting 1) a device comprising: a) a surface, b) a first strand of a nucleobase polymer linked to an area of the surface, c) a second strand of a nucleobase polymer configured to hybridize with the first strand and is hybridized with the first strand forming a duplex, and d) a ligand linked to the second strand; 2) a test compound; and 3) a cell comprising a receptor to the ligand applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the first strand no longer hybridizes to the second strand, the first strand becomes a single stranded nucleobase polymer; contacting the first strand with a Cas nuclease and guide RNA complex and a single stranded reporter nucleic acid, wherein the reporter nucleic acid comprises a fluorescent dye and a quencher conjugated to the reporter nucleic acid in sufficient spatial proximity such that the fluorescent dye is quenched by the quencher; wherein the guide RNA hybridizes with the first strand providing a Cas guide RNA and first strand complex, wherein the Cas guide RNA and first strand complex indiscriminately cleave the reporter nucleic acid such that the fluorescent dye is no longer in close proximity of the quencher providing a fluorescent signal; and detecting the fluorescent signal as an indicator of the ability of the test compound to inhibit receptor binding to the ligand in the presence of the test compound.

In certain embodiments, the method further comprises recording the fluorescent signal on a computer readable medium (non-transient storage medium), e.g., computer hard drive, rewritable mass storage devices, CDROM, RAM, magneto-optical disk, flash drives, solid-state drives (SSDs), non-volatile memory media, SATA drive, nonvolatile memory express (NVMe), and various types of disks, whether locally or on a remote (cloud) server or computer.

In certain embodiments, the method further comprises comparing the fluorescent signal to a normal or reference signal obtained in the absence of the test compound and quantifying the difference and recording the difference on a computer readable medium.

In certain embodiments, this disclosure relates to methods of detecting the ability of platelets to coagulate blood in a sample comprising; contacting 1) a device comprising: a) a surface, b) a first strand of a nucleobase polymer linked to an area of the surface, c) a second strand of a nucleobase polymer configured to hybridize with the first strand and is hybridized with the first strand forming a duplex, and d) a ligand linked to the second strand, wherein the ligand comprises an RGD sequence; 2) a sample from a subject comprising platelets wherein if the platelets present a receptor to the ligand applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the first strand no longer hybridizes to the second strand, the first strand becomes a single stranded nucleobase polymer; contacting the first strand with a Cas nuclease and guide RNA complex and a single stranded reporter nucleic acid, wherein the reporter nucleic acid comprises a fluorescent dye and a quencher conjugated to the reporter nucleic acid in sufficient spatial proximity such that the fluorescent dye is quenched by the quencher; wherein the guide RNA hybridizes with the first strand providing a Cas guide RNA and first strand complex, wherein the Cas guide RNA and first strand complex indiscriminately cleave the reporter nucleic acid such that the fluorescent dye is no longer in close proximity of the quencher providing a fluorescent signal; and detecting the fluorescent signal as an indicator of the ability of the platelet to coagulate blood in a sample.

In certain embodiments, this disclosure relates to methods of detecting the ability of a test compound to inhibit platelet aggregation comprising; contacting 1) a device comprising: a) a surface, b) a first strand of a nucleobase polymer linked to an area of the surface, c) a second strand of a nucleobase polymer configured to hybridize with the first strand and is hybridized with the first strand forming a duplex, and d) a ligand linked to the second strand, wherein the ligand comprises an RGD sequence; 2) a test compound; and 3) a sample from a subject comprising platelets wherein if the platelets present a receptor to the ligand applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the first strand no longer hybridizes to the second strand, the first strand becomes a single stranded nucleobase polymer; contacting the first strand with a Cas nuclease and guide RNA complex and a single stranded reporter nucleic acid, wherein the reporter nucleic acid comprises a fluorescent dye and a quencher conjugated to the reporter nucleic acid in sufficient spatial proximity such that the fluorescent dye is quenched by the quencher; wherein the guide RNA hybridizes with the first strand providing a Cas guide RNA and first strand complex, wherein the Cas guide RNA and first strand complex indiscriminately cleave the reporter nucleic acid such that the fluorescent dye is no longer in close proximity of the quencher providing a fluorescent signal; and detecting the fluorescent signal as an indicator of the ability of the test compound to inhibit receptor binding to the platelets in the presence of the test compound.

In certain embodiments, the test compound is a clotting agent, anti-clotting agent, anti-platelets agent, glycoprotein IIb/IIIa inhibitor, aspirin, clopidogrel, ticagrelor, prasugrel, cilostazol, dipyridamole, tirofiban, eptifibatide (Integrilin™), abciximab (anti-glycoprotein IIb/IIIa receptor antibody), vorapaxar, cilostazol, dipyridamole, warfarin (coumadin), acenocoumarol, phenprocoumon, atromentin, phenindione, a heparin, heparin tetrasaccharide, pentosan polysulfate, phosphomannopentanose sulfate, recombinant factor VIII, recombinant factor VII, tissue plasminogen activator molecule (tPA), urokinase plasminogen activator, factor IIa (dabigatran), or factor Xa (rivaroxaban, apixaban and edoxaban).

In certain embodiments, methods are performed on a sample obtained from a subject at risk of, exhibiting symptoms of, or diagnosed with a clotting or bleeding disorder such as Wisckott Aldrich Syndrome, May-Hegglin anomaly, deep vein thrombosis, pulmonary thrombosis, Factor V Leiden mutation, PT gene mutation, protein C and S deficiency, hemorrhagic stroke, severe bleeding, hemophilia A/B, or von Willebrand's disease.

In certain embodiments, this disclosure relates to methods performed on a sample obtained from a subject at risk of, exhibiting symptoms of or diagnosed with trauma-induced coagulopathy.

In certain embodiments, this disclosure relates to methods of identifying a subject with heparin induced thrombocytopenia comprising 1) contacting a blood sample from a subject with a complex of platelet factor 4 (PF4) and heparin, and optionally exogenous platelets providing a platelet sample mixture; 2) contacting the platelet sample mixture with a device comprising: a) a surface, b) a first strand of a nucleobase polymer linked to an area of the surface, c) a second strand of a nucleobase polymer configured to hybridize with the first strand and is hybridized with the first strand forming a duplex, and d) a ligand linked to the second strand wherein the ligand comprises an RGD sequence; and wherein if the platelets present a receptor to the ligand applying receptor-mediated tension to the device at the receptor opposite the ligand after contacting the platelet sample mixture with the device whereby the receptor binds the ligand providing receptor-mediated tension and the receptor-mediated tension exerts forces exceeding a total force needed to denature the duplex, whereby the first strand no longer hybridizes to the second strand, the first strand becomes a single stranded nucleobase polymer;
contacting the first strand with a Cas nuclease and guide RNA complex and a single stranded reporter nucleic acid,
wherein the reporter nucleic acid comprises a fluorescent dye and a quencher conjugated to the reporter nucleic acid in sufficient spatial proximity such that the fluorescent dye is quenched by the quencher;
wherein the guide RNA hybridizes with the first strand providing a Cas guide RNA and first strand complex,
wherein the Cas guide RNA and first strand complex cleave the reporter nucleic acid such that the fluorescent dye is no longer in close proximity of the quencher providing a fluorescent signal; and
detecting, measuring, or quantifying the fluorescent signal as an indicator of the ability of the platelet to coagulate blood in a sample wherein the detected signal is compared to a normal signal and the signal is higher than the normal signal or comparing the detected signal to a reference signal wherein the reference signal is higher than a normal signal; and correlating the detected signal as an indication that the subject has heparin induced thrombocytopenia; or if the detected signal is normal that the subject does not have heparin induced thrombocytopenia or if the detected signal is lower than reference signal indicating heparin induced thrombocytopenia that the subject does not have heparin induced thrombocytopenia.

In certain embodiments, any methods disclosed herein may further comprise the steps of recording the detection, measurement, quantification or diagnosis on a computer readable medium (non-transitory), and optionally communicating the detection, measurement, quantification or diagnosis to a medial professional or the subject/human patient. In certain embodiments, the subject is a human patient at a hospital or emergency room.

In certain embodiments, methods are performed on a sample obtained from a subject, such as a human patient, to predict bleeding risk before, during and after surgery. In certain embodiments, methods are performed on a sample obtained from a subject before, during, or after surgery to determine whether the patient is at risk of coagulopathy or blood clotting before, during, or after surgery.

In certain embodiments, methods are performed on a sample obtained from a subject having a condition or surgical intervention selected from acute coronary syndrome, percutaneous coronary intervention (PCI), stenting, mechanical heart valve, ischemic stroke, peripheral arterial disease, device closure of an atrial septal defect (ASD), stable angina, coronary artery bypass grafting surgery, thrombocytosis, coronary artery disease, colon cancer, Kawasaki disease, rheumatic disease, patent ductus arteriosus (PDA) device closure, pericarditis, atrial fibrillation, stroke, or venous thromboembolism.

In certain embodiments, methods are performed on a sample obtained from a subject before, during, or after surgery to reduce the duration of bleeding or to identify and administer therapies to prevent or reduce onset of or duration of bleeding.

In certain embodiments, methods are performed on a sample obtained from a subject before, during, or after surgery to reduce blood clotting or to identify and administer therapies to prevent or reduce onset of or duration of blood clotting or excessive bleeding.

In certain embodiments, methods are performed on a sample obtained from a subject before, during, or after surgery to determine whether the patient is at risk of coagulopathy or sever bleeding before, during, or after cardiac surgery.

In certain embodiments, methods are performed on a sample obtained from a subject connected to life support system/device that incorporates blood transfusions to determine whether the patient is at risk of coagulopathy, blood clotting, or sever bleeding before, during, or after being connected to the life support system device. In certain embodiments, the device provides extracorporeal membrane oxygenation to the blood. In certain embodiments, the device is a hemodialysis machine. In certain embodiments, the subject is diagnosed with liver or kidney disease.

In certain embodiments, methods are performed on a sample obtained from a subject that is a premature baby, less than one or two years old, less than 16 years old, over 55 or 65 years old or any children and adults with a heart condition or disease and/or lung condition or disease.

In certain embodiments, methods are performed on a sample obtained from a subject that is diagnosed with cancer, leukemia, lung cancer, lung viral infection, lung bacterial infection, bronchiectasis, or cystic fibrosis. In certain embodiments, methods are performed on a sample obtained from a subject that is at risk of, exhibiting symptoms of, or diagnosed with a coronavirus, influenza virus, adenovirus, enterovirus, or respiratory syncytial virus.

In certain embodiments, the subject is diagnosed with hemophilia A or B or the subject is diagnosed with acquired hemophilia or thrombocytopenia.

### Mechano-Cas12a Assisted Tension Sensor (MCATS) Assay and Advantages

A Mechano-Cas12a Assisted Tension Sensor (MCATS) assay was developed. Cas12a nuclease is used in an ultrasensitive fluorescence-based assay to detect the molecular forces generated by cells. CRISPR-Cas12a (Cpf1) is a class 2 type V-A enzyme that binds with a single-stranded guide CRISPR RNA (crRNA). The crRNA-loaded Cas12a can be activated using two different approaches. The first is by binding to a complementary target double stranded DNA (activator) containing a PAM sequence TTTV for LbCas12a. Double stranded DNA lacking the PAM is a poor activator of Cas12a. Alternatively, Cas12a can be activated by binding to a complementary target single stranded activator DNA. In this case, the PAM sequence is not required to potently activate the nuclease. Upon activation of the Cas12a, the enzyme undergoes a conformation change that unleashes its indiscriminate cleavage activity which hydrolyzes any ssDNA in proximity.

In the MCATS assay, the activator is a ssDNA (that lacks the PAM sequence) and is anchored to a surface, such as a glass slide. The activator is concealed by hybridization to a complementary strand that is in turn conjugated to a peptide such as, cyclo-Arg-Gly-Asp-(d)Phe-Lys (cRGDFK, SEQ ID NO: 1), or any protein ligand specific to the cell receptor of interest (FIG. 1A-B). When cells are seeded on this surface, surface receptors such as integrins bind to the cRGDFK (SEQ ID NO: 1) ligand on the duplex and apply forces. Forces that exceed the mechanical tolerance of the duplex lead to its rupture, exposing the activator (bottom strand) and thus triggering Cas12a nuclease activity. Upon activation, Cas12a will indiscriminately and catalytically cleave fluorogenic single stranded DNA reporter for amplification. Because Cas12a is highly efficient its activation generates a massive fluorescence signal output, that can be measured using a conventional fluorometer or plate reader for facile and high throughput readout.

Aggregometry (measuring platelet aggregation) and thromboelastography (TEG, measuring the viscoelastic properties of a clots) assays are used to measure platelet function. Platelet function and aggregation is highly dependent on platelet count. Patients with higher platelet counts will tend to form aggregates more readily regardless of the functional activity of each individual platelet thus limiting the predictive value in identifying post operation bleeding complications, e.g., in the context of cardiopulmonary bypass patients, due to limited specificity and a high number of false positive and false negative readings. Conventional tools for measuring platelet function have limited specificity and sensitivity and require relatively large blood volumes that prohibit wider drug screen testing.

In certain embodiments, this disclosure contemplates using devices and methods disclosed herein for measuring platelet function to fine tune anti-platelet drug choices and optimal dosing. Each patient responds to anti-platelet agents differently because of multiple factors including concomitant medications, genetic differences, health condition, and patient non-compliance.

The heterogeneous response to anti-platelet therapy is well documented. For example, ⅓ of all Americans over the age of 40 are on a low dose aspirin regiment due to its ability to reduce incidence of stroke. But about 4.7% and 21% of acute coronary syndrome patients were found to be resistant to aspirin and clopidogrel (Plavix™), respectively. It would be highly desirable to use greater dosing or to use an alternate treatment or intervention plan for patients resistant to a specific therapy. Even the same patient can display variability in response to anti-platelet therapy due to a secondary infection or other factors such as emotional and physiological factors. Finally, a sub-set of patients do not adhere to prescribed dosing and again this creates complexities for patient care. Taken together, there is a significant need to develop specific and sensitive assays to faithfully measure platelet function.

MCATS platform can be used to quantify platelet forces with the goal of assessing platelet dysfunction for patients undergoing cardiopulmonary bypass (CPB) surgery. Almost a quarter of CPB surgeries lead to severe postoperative bleeding requiring blood transfusion. The ability to anticipate the risk for uncontrolled bleeding is highly desirable. One could administer blood transfusions more rapidly and to the patients that need it the most.

MCATS was used to measure platelet function in donors. These experiments indicated that their tension signal was similar. Following CPB donors, the platelet tension signal using the MCATS assay dropped, and this coincided with the need to administer platelet transfusions for these donors. Importantly, TEG measurements post-CPB indicated normal platelet function for one of the donors that needed transfusion underscoring the poor sensitivity of the TEG assay.

Because MCATS can be performed at volumes of around 5 microliters (uL) of blood or less to conduct each measurement, a typical blood draw (5 mL) allows one to run many assays in a rapid manner. This allows for the capability to screen the activity of a panel of clinically approved anti-platelet drugs such as aspirin, eptifibatide (Integrilin™), and abciximab (anti-glycoprotein IIb/IIIa receptor antibody) for donors. MCATS to be used for personalized tailoring of anti-coagulant drugs. An accurate, rapid, and cost-efficient method to detect molecular forces opening the door for biochemical lab to access cell mechanics in biological and pathological studies.

Typical tools for measuring platelet function are based on aggregometry and viscoelastic assays. Aggregometry measures the light transmission of platelet rich plasma sample (about 1 mL) using a dedicated instrument that applies shear as well a specific agonist such as ADP and TRAP. Aggregometry records multiple parameters including the kinetics of aggregation to infer clotting functions. Similarly, TEG uses a dedicated instrument that records the drag forces in whole blood samples which measure the kinetic information about clot formation and viscoelastic properties of clots.

TEG and aggregometry assays require dedicated instruments which limits widespread dissemination. PFA and TEG require about mL quantities of blood for each measurement. This prohibits running triplicates and performing drug screens. TEG and PFA assays have limited sensitivity given that these assays require platelet aggregation, which is highly dependent on platelet count, coagulation factors such as fibrinogen level, and usage of antiplatelets agents. Patients with higher platelet counts will tend to form more stable aggregates regardless of the functional activity of each individual platelet.

In testing MCATS, a patient with a low platelet count, which would present a significant challenge for TEG, did not impact the MCATS assay. A unique merit of MCATS is that it measures molecular forces directly using conventional plate reader and it uses Cas12a amplification to boost the signal for fast and sensitive readout. As a result, MCATS uses only about 5 μL of blood or less, and hence a typical blood draw (5 mL) is enough to run 1000 assays.

Antiplatelet therapies are commonly prescribed to prevent acute arterial thrombosis. However, antiplatelet therapy assessment based on aggregometry has not shown improved outcomes and better benefit/risk for cardiovascular patient. MCATS can generate personalized dose-response curves for specific drugs rapidly to optimize treatment in a dynamic manner, which may increase the sensitivity and specificity required to guide personalized platelet therapy. The forces applied by GPIIb/IIIa were measured which mediates platelet adhesion and aggregation to assess platelet functions. Other platelet forces mediated by GPIb-IX-V and GPVI can potentially be investigated by attaching different ligands/proteins on DNA tension probe to understand the platelet forces with different receptors under shear flow or in different hemostasis states. Live and active cells are used to perform MACTS assay. Lyophilized platelets (fixed platelets with intact protein structure) failed to generate MCATS signal.

In MCATS experiments, different platelet handling may lead to significantly different platelet response. Therefore, different platelet purification procedures were tested, and the results indicated that MCATS can detect tension signal from purified platelets, platelet rich plasma and whole blood sample. However, whole blood showed decreased tension signal in MCATS because the high abundance of red blood cells blocked access to the chip surface. It is contemplated that applying mild flow conditions would better allow performing MCATS in whole blood.

The MCATS assay can also be performed on platelet rich plasma (PRP). PRP showed slightly decreased MCATS signal compared to that of purified platelets. Advantages of using PRP include simplifying blood processing steps and decreasing preparation time to run the assay. Other advantages of using PRP include maintaining the physiological drug dose found in the blood which is important for assessing drugs that have short half-life or rapid off-rate. Conversely, the use of purified platelets for MCATS enhanced the signal and provided a more direct evaluation of platelet function without interference from soluble factors.

Another parameter to consider in MCATS is the assay time. MCATS signal in the platelets and fibroblasts experiments were monitored with different amplification times. The results showed that 30 min amplification provides sufficient signal while 60 min reaction time provides enhanced signal for better sensitivity. Further increasing amplification time does not lead to significantly improved signal. These results indicate that MCATS applications can potentially utilize 30 min amplification in a more rapid assay or use 60 min amplification for higher sensitivity.

MCATS is an ultrasensitive fluorescence-based assay that rapidly measures cell receptor mediated tension. When cell receptors apply sufficient force, the DNA duplex tension sensor denatures exposing single stranded DNA. The peeled DNA is amplified using Cas12a to produce about 10⁴ fluorophores in response to each 12 pN event, which allows rapid (about 1-2 hrs) high-throughput detection of cellular tension without requiring any dedicated hardware. Instead, the assay uses modified 96 well plates that are read out in a conventional plate reader. The MCATS platform can be used to detect platelet dysfunction which may predict bleeding risk and the need for platelet transfusion in CPB patients. High throughput detection of platelet forces provides a more integrative and accurate measurement of platelet function.

### Testing Parameters of MCATS Assay

Considering that immobilization of a single stranded nucleic acid activator imposes a steric constraint, the kinetics of Cas12a cleavage activity was tested when the activator is immobilized on a surface. In this assay, a biotinylated single stranded nucleic acid activator was incubated on streptavidin-coated surfaces. The single stranded activator or double stranded (concealed) activator were immobilized on surfaces by incubating the biotinylated derivative in 100 nM solutions for 1 hr at room temperature (RT). This procedure typically leads to an activator density of about 1330±60 molecules/μm².

The Cas12a-gRNA complex and single strand reporter DNA conjugated to a fluorescent quencher pair were added to the surface and a fluorescence plate reader was used to monitor the fluorescence signal in each well of the 96 well plate. Fluorescent measurements showed that single stranded activator triggered the Cas12a nuclease functions and generated a strong fluorescent signal. In contrast, the double stranded activator (concealed) only showed minimal signal.

Experiments were performed to determine whether adding a linker to the activator may boost Cas12a cleavage rates. Tethered macromolecules, such as DNA, occupy a large footprint on the surface and hence may reduce the effective activator density. Four types of activators included polyT linkers of 0, 6, 60, and 160 nucleotides were tested. The initial rate constant for these activators was plotted. The initial rate constant was enhanced with longer linkers with the exception of the 160 nt activator. The spacer with 6 nucleotides separates the Cas12a from the surface and hence reduces steric hinderance and potential surface-induced denaturation effects. The 60 nt linker further shows enhanced activity likely because of the flexibility of the Cas12 which allows for auto-cleavage and release from the surface. The 160 nt activator showed the least Cas12a activity, and this is likely due to low surface density of the activator coupled with the potential for truncations and inefficient Cas12a binding. Subsequent work with MCATS employed the 60T linker due to is superior signal amplification.

### Table 1 Shows a List of Oligonucleotides

| Name | Sequence (5′ to 3′) |
|---|---|
| No Spacer Cas12a TGT | /5AmMC6/CC TCC TCA AGG AAC AAC ATT GC/3Bio/ |
| Btm Strand | SEQ ID NO: 3 |
| Cas12a TGT Top Strand | /5Hexynyl/GC AAT GTT GTT CCT TGA GGA GG/3BHQ_2/ |
| | SEQ ID NO: 4 |
| 6T Spacer Cas12a TGT | /5AmMC6/CC TCC TCA AGG AAC AAC ATT GCT TTT TT/3Bio/ |
| Btm Strand | SEQ ID NO: 5 |
| 60T Spacer Cas12a TGT | /5AmMC6/GT GTC GTG CCT CCG TGC TGT GTT TTT (TTT)19T/3Bio/ |
| Btm Strand | SEQ ID NO: 6 |
| ATTO 565N Reporter | /5ATTO565N/TT ATT /3BHQ_2/ |
| DNA | |
| FAM reporter DNA | /56-FAM/TT ATT /3IABkFQ/ |
| TGT crRNA | UAA UUU CUA CUA AGU GUA GAU GCA AUG UUG UUC CUU GAG GA |
| | SEQ ID NO: 2 |

Cas12a activity was measured as a function of assay temperature, buffer, and reaction time. The signal to noise ratio (S/N) of the assay was compared using two fluorogenic ssDNA substrates. The limit of detection (LOD) of assay was investigated for both for surface tethered activator as well as soluble activator as a reference. To tune the activator surface density, surfaces were created comprised of a binary mixture of ssDNA activator with the blocked (double stranded) activator. A total activator solution was maintained at a concentration of 100 nM. The ratio between the blocked and single stranded activator was adjusted. Cas12a-gRNA and fluorogenic reporter were added to the well and the final fluorescence intensity was measured after 1 h of enzyme activity. The LOD was then inferred from the ratio of 3.3×standard deviation of the background normalized by the slope of the fluorescence versus concentration plot. The data indicates that the LOD for nucleic acid sensing in solution was 21 fM and 560 molecule/mm² on a surface. This analysis provides the basis for using MCATS to sensitively detect molecular forces generated by cells.

### Using MCATS to Detect Integrin-Mediated Cell Traction Forces in Cell Lines and Evaluate Drug Interactions

Integrins are a family of heterodimeric cell surface adhesion receptors that bridge the cellular cytoskeleton with the extracellular matrix (ECM) to mediate a variety of processes including cell adhesion, and coagulation. Integrin receptors can apply pN forces which are sufficient to mechanically denature DNA duplexes. The MCATS assay was used to measure integrin receptor forces with NIH/3T3 cells.

DNA duplexes that present cRGDFK (SEQ ID NO: 1) ligand and concealed Cas12a activator were immobilized on the surface. The conjugation of ligand to duplex was achieved via copper(I)-catalyzed azide-alkyne cycloaddition and was verified with ESI mass spectroscopy. NIH/3T3 fibroblast cells were then seeded on these surfaces for 1 hr.

Two types of DNA duplexes were designed having identical sequence and thermal melting temperatures, but different geometries and mechanical tolerances (FIG. 2B). When the activator is anchored through its 5′ terminus, the cRGDFK (SEQ ID NO: 1) ligand is presented on the 3′ terminus of the top strand. Hence this probe denatures through an unzipping process which has a lower activation barrier and displays a mechanical rupture threshold of about 12 pN. In contrast, when the activator is anchored through its 3′terminus, the probe will denature by shearing which has a larger mechanical threshold of about 56 pN. The same number of cells (25,000 cells) were incubated on the two types of surfaces for 1 hr before running the Cas12a amplification assay. An increase in fluorescence signal was observed for both the 12 pN and 56 pN probes (tagged with fluorophore quencher pairs) due to mechanical denaturation of the duplex. The probes in the unzipping geometry (12 pN) were more significantly denatured compared to shearing mode probes (56 pN). Cas12a and reporter DNA were added to trigger the MCATS assay and allowed the reaction to proceed for 1 hr and then measured bulk fluorescence (540 nm excitation and 590 nm emission) using the plate reader (FIG. 2C). Importantly, the 12 pN unzipping mode probes also generated a greater MCATS signal, reflecting the greater density of exposed activator. MCATS produced over 100-fold greater signal compared to mechano-HCR which confirms that this assay is more sensitive and offers a simplified experimental process as no washing steps are required. The MCATS assay was validated by titrating increasing numbers of NIH/3T3 cells in 96 well plates and measuring associated MCATS signal in each well. Increasing cell densities led to greater MCATS signal. MCATS can detect as little as 50 fibroblasts in a 96 well plate using a plate reader.

Considering that receptor mediated tension plays an important role in cell function, quantifying molecular forces of cell in a rapid fashion facilitates applications such as screening dose-response curves for drugs that modulate cell mechanics. Experiments were performed to determine whether MCATS can be used to produce a dose response curve for NIH-3T3 cell incubated with Rho kinase inhibitor, Y-27632, which targets the phosphorylation of myosin light chain and therefore dampens forces transmitted by focal adhesions. NIH/3T3 cells were pretreated with a range of Y-27632 concentrations (0-50 μM) for 30 min. MCATS was ran with the drug treated cell. The MCATS signal showed a dose-dependent reduction as a function of increasing Y-27632 concentration, indicating that plate-reader based MCATS readout can report cell forces modulated by MLC inhibition. By fitting the data to a standard dose-response inhibition function (signal=100/(1+[drug]/IC₅₀)), the mechano-IC₅₀=7.9 M (95% CI=5.5-11.6 μM), was calculated and consistent with previously reported IC₅₀ values of about 5-10 M.

Using MCATS to Detect the Mechanical Signal of Human Platelets with Antiplatelet Drugs.

Platelets are primary cells wherein contractile forces are integral to the function of platelets in forming clotting, resisting blood shear flow, and sealing wounds. Anti-platelet agents are some of the most commonly prescribed drugs. The agents are sometimes ineffective or have risks such as abnormal bleeding as a side effect. There are different types of antiplatelet drugs. Aspirin inhibits the activity of cyclooxygenase (COX) and prevents formation of thromboxane A2 and thus functions as an anti-coagulation agent. Integrin αIIbβ3 antagonist, eptifibatide, and 7e3, which is an antibody that binds to glycoprotein (GP) IIb/IIIa receptor, prevent platelets aggregation.

Platelet-rich plasma (PRP) is prepared by centrifugation and separation from whole blood. Then platelets rich plasma was separated and centrifuged a second time with apyrase and resuspended in Tyrode's solution, which is isotonic with interstitial fluid, contains magnesium, and uses bicarbonate and phosphate as a buffer. Apyrase is important in the platelets purification process to prevent hemolysis caused platelets aggregation. MCATS was tested as a function of the number of platelets seeded on the Tₜₒₗ=12pN surface (FIG. 3A). MCATS detected as few as 2000 platelets. About 2×10⁶ human platelets were used in the following experiments because this concentration of platelets in a 96-well plate offered a strong signal for the assay. In a second set of experiments, platelets were incubated with different drugs at room temperature for 30 minutes and plated 2×10⁶ treated human platelets in each well for 1 h and then performed MCATS.

For all the compounds tested, a dose-dependent decrease in the 12 pN duplex rupture was observed in both microscopy images and MCATS signal read out with a plate reader. By fitting the plot of MCATS signal with standard dose-response inhibition function (Signal=Bottom+(Top−Bottom)/(1+([drug]/IC₅₀)), the mechanical IC₅₀ for aspirin, eptifibatide and 7E3 was determined for each individual donor and the data was plotted in FIGS. 3B and 3C. MCATS was found to be robust as the signal generated from the same blood draw from the same donor was highly consistent (sigma about 5%). However, donor-to-donor variability was found in mechano-IC₅₀ which likely reflects the biological heterogeneity of the drug response as well as the inherent differences in platelet-to-platelet activity. The measured values were consistent with other reports. A benefit of MCATS is that each evaluation only requires 2×10⁶ platelets while 1 mL of blood usually contains about 10′ platelets. Therefore, a typical 5 mL blood draw can be used to run hundreds of assays and screen different type of drugs in a single run.

### Using MCATS to Assess Platelets Dysfunction and Predict Bleeding Risk in Patients Undergoing Surgery

MCATS measurement were obtained on 2 healthy donors as well as 2 patients undergoing cardiopulmonary bypass (CPB) surgery, bot pre- and post-operation. Samples were also tested using Thromboelastography (TEG) as a standard benchmark to compare against MCATS. Patients before surgery and samples from healthy donors were used as a baseline. The CPB patient pre-surgery (n=2) and healthy donors (n=2) showed similar MCATS tension signal. However, after CPB surgery, both patients that underwent surgery showed a significant decrease in MCATS signals. This decrease was consistent with the clinical need to administer blood transfusions to both patients. The TEG for one of the CPB patients showed a normal result both pre- and post-operation. The mechano-IC₅₀ of aspirin was also tested for the healthy donor and CPB donors before and after surgery. The results showed that the surgery did not influence the mechano-IC₅₀ significantly (FIG. 4B). Another parameter that could influence platelet forces is ADP agonist which is well known to trigger platelet activation and adhesion. the dose-response to agonist using MCATS was tested. Increasing tension signal as a function of ADP was found (FIG. 4C). These results were further validated with light transmission aggregometry.

### Surface Preparation

MCATS surface preparation method was performed using rectangular glass coverslips (25 30×75 mm,) rinsed with water and sonicated for 20 minutes in water and 20 minutes in ethanol. The glass coverslips were then cleaned with piranha solution. The piranha solution was prepared using a 1:3 mixture of H₂O₂ and H₂SO₄. Then, slides were washed 6 times in beakers with ultrapure water, followed by 4 successive washes using ethanol. In a separate beaker of ethanol, slides were reacted with 3% v/v APTES at room temperature for 1 h. Coverslips were then washed 6 times with ethanol, baked in oven for 20 minutes at 80° C. Slides were then reacted with NHS-PEG-biotin (3% w/v) for 1 hr in ultrapure water. Next, slides were washed 3 times with ultrapure water, dried under N₂ gas, and then stored at −30° C. for up to 2 weeks before use. At the day of imaging, the 5 kDa PEG-biotin surface was adhered onto microtiter plate with an adhesive bottom. Wells were then reacted with 50 μg/mL streptavidin for one hour. The wells were then washed with 1×PBS and incubated with 100 nM DNA probe solutions for 1 hr. Finally, the wells were washed with 1×PBS.

DNA Hybridization and gRNA/Cas12a Binding

DNA oligonucleotides were hybridized at 100 nM in a 0.2 μL PCR tube. DNA was first heated to 90° C. and then cooled at a rate of 1.3° C. per min to 35° C. gRNA and Cas12a were incubated for 10 min at 37° C. at 20 nM in a 0.2 μL PCR tube just before adding onto a surface and stored on ice for maximum preservation of activity.

### Oligo Dye/Ligand Coupling and Purification

Cyclic c(RGDFK (SEQ ID NO: 1)(PEG-PEG)) (100 nmoles) was reacted with about 200 nmoles of NHS-azide in DMSO overnight. Product was then purified via reverse phase HPLC using a C18 column (Solvent A: water-+0.05% TFA, SolventB: acetonitrile+0.05% TFA; starting condition: 90% A+10% B, 1%/min; Flow rate: 1 mL/min). Purified product was ligated to the BHQ2 top strand via 1,3-dipolar cycloaddition reaction. An alkyne ligand strand (5 nmoles) was reacted with about 70 nanomoles of product. The total reaction volume is 50 μL, composed of 0.1 M sodium ascorbate and 0.1 mM Cu-THIPTA for 2 h at room temperature. The product was then purified with a P2 size exclusion column, and then purified with reverse phase HPLC oligo column (Solvent A: 0.1M TEAA, Solvent B: acetonitrile; starting condition: 90% A+10% B, 0.5%/min gradient B, Flow rate: 0.5 mL/min).

Amine labelled hairpin strands (10 nmole) were reacted overnight with 20 times excess of Cy3B-NHS or ATTO 647N-NHS dissolved in 10 μL DMSO. The total volume of the reaction was 100 μL, composed of 1×PBS supplemented with 0.1M NaHCO₃. Then P2 size exclusion gel was used to remove unreacted dye. The product was then purified by reverse phase HPLC using oligo column (Solvent A: 0.1M TEAA, Solvent B: acetonitrile; starting condition: 90% A+10% B, 1%/min gradient B, Flow rate: 0.5 mL/min) to purify products.

### Solution Based Cas12a Amplification and Plate Reader Readout

Cas12a reactions were performed at 37° C. for 1 h. A total activator solution concentration of 100 nM was maintained but the ratio was tuned between the blocked and single stranded activator. A combined 20 nM Cas12a-gRNA complex and 100 nM fluorogenic reporter were then added to the wells. The final fluorescence intensity was measured using a plate reader with filter set (Ex/Em=540/590 nm for reporter channel) during 1 h of enzyme activity.

### Human Platelet Handling and Ethics Agreement

Blood was collected and anticoagulated with sodium citrate or EDTA. The sample was then centrifuged for 12 min at 140 g (with 0.02 U Apyrase). Then platelets rich plasma was separated and centrifuged for 5 min at 700 g with 3 uM PGE-1 and 0.02 U Apyrase. The platelets were then resuspended in Tyrode's buffer with 3 uM PGE-1 and centrifuged for 5 min at 700 g. Platelets were resuspended in Tyrode's buffer. It is worth noting that apyrase is important in the platelets purification process to prevent hemolysis caused by aggregation of platelets.

### Mechano-Cas12a Assisted Tension Sensor

MCATS assay was performed on the prepared biotin surface as described in the surface preparation section. First, hybridized concealed activator probes were incubated on biotin surface in 1×PBS buffer for 1 h. The wells were washed with 1×PBS. Then, cells were added onto the cRGDFK(SEQ ID NO: 1)-labelled duplex surfaces for 1 h to promote cell adhesion with DMEM supplemented with 1% serum for NIH/3T3 cells or Tyrode's buffer with 10 μM ADP for platelets. Surfaces with exposed activator were imaged directly with fluorescence microscopy for high resolution characterization and quantification. Subsequently, gRNA/Cas12a complex and reporter DNA were mixed and added to the well to initiate the Cas12a amplification reaction with mechanically exposed activator. After 1 h of incubation, fluorescence intensities on 96 well plates were measured with a plate reader (Ex/Em=540/590 nm for reporter channel).

### Dose-Dependent Inhibition of Receptor Mediated Tension

For dose-dependent inhibition of experiments, the cell density of 3T3 fibroblasts was first characterized with a hemocytometer. Same number of 3T3 cells (25000) in cell culture medium were then incubated with different concentrations of inhibitor in the cell culture incubator for 30 minutes before plating onto 96 well plates. Afterwards, cells were incubated for 1 h to promote cell adhesion. Then the MCATS protocol was followed to achieve amplification and quantification. For platelet MCATS measurements, human platelets were purified and incubated at room temperature for at least 30 min before beginning experiments. Platelets were then treated with drugs for 30 min before seeding onto 96 well-plates. Ten (10) μM ADP was added to promote cell adhesion. Then platelets were incubated at room temperature for 1 h. The same MCATS protocols were followed to achieve amplification and quantification for platelets.

### Uses in Detecting Heparin Induced Thrombocytopenia (HIT)

The use of heparin also comes with a risk of heparin induced thrombocytopenia (HIT). The pathogenesis begins with antibody formation to a complex of heparin and a platelet protein called PF4. These HIT antibodies can then bind and activate platelets causing thrombosis in blood vessels. Once suspected, heparin must be stopped, an alternative anticoagulant administered. HIT antibodies present in the blood of the patient can be detected via a screening ELISA assay. Since most antibodies are not functional and capable of activating platelets. The second step in testing is to determine whether the antibodies are functional using a serotonin release assay (SRA).

Serum from donors that are SRA positive and SRA negative were obtained. Healthy donor platelets were treated with this serum in the presence of PF4 and low concentration heparin to mimic the pathogenic HIT response. After the samples were incubated for a few minutes, the MCATS assay was performed. One is able to detect the miniscule forces generated by a single platelet when it is activated by HIT antibodies. When the platelet receptors apply sufficient force, the DNA duplex “peels” exposing single stranded DNA. The peeled DNA was amplified using CRISPR technology to produce about 10⁴ fluorophores in response to each pN event. The fluorescent signal can then be measured. The whole assay required less than 1 hr. The results show that MCATS can distinguish between HIT positive and HIT negative patients from this limited pilot data set. Note that the SRA+/− serum from 14 donors was used to activate platelets from three healthy donors and hence signal is sufficiently robust to overcome biological heterogeneity.

What is claimed is:

1. A method of identifying a receptor binding to a ligand comprising;
contacting 1) a device comprising:
a) a surface,
b) a first strand of a nucleobase polymer linked to an area of the surface,
c) a second strand of a nucleobase polymer configured to hybridize with the first strand and is hybridized with the first strand forming a duplex, and
d) a ligand linked to the second strand; and
2) a cell comprising a receptor to the ligand applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the first strand no longer hybridizes to the second strand and the first strand becomes a single stranded nucleobase polymer;
contacting the first strand with a Cas nuclease and guide RNA complex and a single stranded reporter nucleic acid,
wherein the reporter nucleic acid comprises a fluorescent dye and a quencher conjugated to the reporter nucleic acid in sufficient spatial proximity such that the fluorescent dye is quenched by the quencher;
wherein the guide RNA hybridizes with the first strand providing a Cas guide RNA and first strand complex,
wherein the Cas guide RNA and first strand complex cleave the reporter nucleic acid such that the fluorescent dye is no longer in close proximity of the quencher providing a fluorescent signal; and
identifying the receptor binding to a ligand by detecting the fluorescent signal as an indicator of the receptor binding to the ligand.

2. The method of claim 1 wherein the Cas guide RNA and first strand complex cleave a single-stranded segment of the first strand de-conjugating the complex from the surface.

3. The method of claim 1 wherein the first stand comprises a fluorescent dye or quencher, wherein the second strand comprises a fluorescent dye or quencher, and wherein when the second strand is hybridized with the first strand forming a duplex the associated fluorescent dye and quencher are in sufficient spatial proximity such that the fluorescent dye is quenched by the quencher.

4. The method of claim 1, wherein the ligand comprises a polysaccharide, peptide, glycopeptide, or steroid structure.

5. The method of claim 1, wherein the first strand comprises a protospacer-adjacent motif (PAM) associated with the Cas nuclease and guide RNA complex.

6. The method of claim 1 wherein the Cas nuclease is Cas12a.

7. The method of claim 1, wherein the surface is a transparent glass or polymer.

8. The method of claim 1, wherein contacting the device and the cell is at a temperature of greater than 20, 30, or 35 degrees Celsius.

9. The method of claim 1, wherein first strand of a nucleobase polymer is linked to an area of the surface that does not hybridize to the second strand that is of a length of less than 160, 150, or 100 nucleotides.

10. The method of claim 1, wherein contacting the device and the cell is in the presence of a growth medium with exogenously added magnesium (Mg) salt.

11. The method of claim 10, wherein the exogenously added magnesium salt is at a concentration of greater than 1 mM.

12. The method of claim 1 wherein denaturing the duplex is by sequentially dehybridizing individual base pairs (unzipping) or non-sequential dehybridization (shearing).

13. The method of claim 1, further comprising recording the fluorescent signal on a computer readable medium providing a background signal, adding a test compound to the device, detecting the fluorescent signal on a computer readable medium providing a test compound signal, and comparing the background signal to the test compound signal.

14. The method of claim 1, wherein the ligand is a peptide comprising an RGD sequence.

15. The method of claim 14 wherein the peptide is of the following formula: 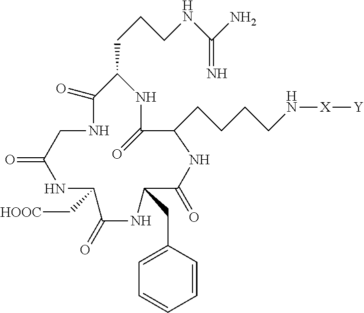
wherein X is a linking group Y is the second strand nucleobase polymer.

16. A composition of matter comprising the RGD peptide having the following formula: 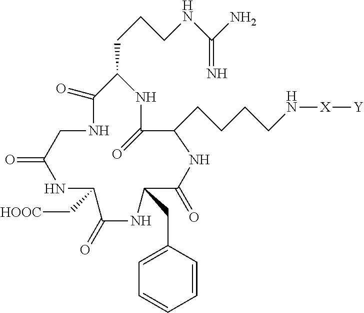
or salt thereof, wherein X is a linking group and Y is a nucleobase polymer.

17. A method of identifying the effect of a test compound to inhibit a receptor binding to a ligand comprising;
contacting 1) a device comprising:
a) a surface,
b) a first strand of a nucleobase polymer linked to an area of the surface,
c) a second strand of a nucleobase polymer configured to hybridize with the first strand and is hybridized with the first strand forming a duplex, and
d) a ligand linked to the second strand;
2) a test compound; and
3) a cell comprising a receptor to the ligand applying receptor-mediated tension to the device at the receptor opposite the ligand after the contacting step whereby the receptor binds the ligand providing receptor-mediated tension and the receptor-mediated tension exerts forces exceeding a total force needed to denature the duplex, whereby the first strand no longer hybridizes to the second strand, the first strand becomes a single stranded nucleobase polymer;
contacting the first strand with a Cas nuclease and guide RNA complex and a single stranded reporter nucleic acid,
wherein the reporter nucleic acid comprises a fluorescent dye and a quencher conjugated to the reporter nucleic acid in sufficient spatial proximity such that the fluorescent dye is quenched by the quencher;
wherein the guide RNA hybridizes with the first strand providing a Cas guide RNA and first strand complex,
wherein the Cas guide RNA and first strand complex cleave the reporter nucleic acid such that the fluorescent dye is no longer in close proximity of the quencher providing a fluorescent signal; and
detecting the fluorescent signal as an indicator of the ability of the test compound to inhibit receptor binding to the ligand in the presence of the test compound.

18-20. (canceled)
